SEQUENCE LISTING (53 sequences)

SEQ ID NO: 1 - protein, 240 aa, synthetic construct
MAGPPRLLLLPLLLALARGLPGALAAQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALPAALAVISFLLGLGLGVACVLARTQIKKLCSWRDKNSAACVVYEDMSHSRCNTLSSPNQYQ

SEQ ID NO: 2 - protein, 155 aa, synthetic construct
AQEVQQSPHCTTVPVGASVNITCSTSGGLRGIYLRQLGPQPQDIIYYEDGVVPTTDRRFRGRIDFSGSQDNLTITMHRLQLSDTGTYTCQAITEVNVYGSGTLVLVTEEQSQGWHRCSDAPPRASALPAPPTGSALPDPQTASALPDPPAASALP

SEQ ID NO: 3 - protein, 10 aa, synthetic construct
AQEVQQSPHC

SEQ ID NO: 4 - protein, 18 aa, synthetic construct
CEDGVVPTTDRRFRGRID

SEQ ID NO: 5 - protein, 11 aa, synthetic construct
TEEQSQGWHRC

SEQ ID NO: 6 - protein, 13 aa, synthetic construct
HRLQLSDTGTYTC

SEQ ID NO: 7 - protein, 10 aa, synthetic construct
CPPTGSALPD

SEQ ID NO: 8 - DNA, 348 bp, synthetic construct
  source 1..348: mol_type = other DNA
gtccagctgcagcagtcaggacctgagctggtgaagcctggggcttcagtgaagatatcctgcaaggcttctggttattcattcactgactacatcatgctctgggtgaagcagagccatggacagagccttgagtggattggaaatattgatccttactatggtagtactggctacaatctgaagttcaagggcagggccacattgactgtcgacaaatcttccagcacagcctacatgcagctcaacagtctgacatctgaggactctgcagtctattactgtgcacgggatggtaactacggaagtgactactggggccaagggaccacggtcaccgtctcctca

SEQ ID NO: 9 - protein, 116 aa, synthetic construct
VQLQQSGPELVKPGASVKISCKASGYSFTDYIMLWVKQSHGQSLEWIGNIDPYYGSTGYNLKFKGRATLTVDKSSSTAYMQLNSLTSEDSAVYYCARDGNYGSDYWGQGTTVTVSS

SEQ ID NO: 10 - DNA, 363 bp, synthetic construct
  source 1..363: mol_type = other DNA
gacattgtgatgacacagtctccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccattcacgttcggctcggggacaaagttggaaataaaacgggctgatgctgcaccaactgtatcc

SEQ ID NO: 11 - protein, 121 aa, synthetic construct
DIVMTQSPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRADAAPTVS

SEQ ID NO: 12 - protein, 7 aa, synthetic construct
GYSFTDY

SEQ ID NO: 13 - protein, 6 aa, synthetic construct
DPYYGS

SEQ ID NO: 14 - protein, 8 aa, synthetic construct
DGNYGSDY

SEQ ID NO: 15 - protein, 16 aa, synthetic construct
RSSQSIVHSNGNTYLE

SEQ ID NO: 16 - protein, 7 aa, synthetic construct
KVSNRFS

SEQ ID NO: 17 - protein, 9 aa, synthetic construct
FQGSHVPFT

SEQ ID NO: 18 - protein, 290 aa, synthetic construct
MALPVTALLLPLALLLHAARPGSGDIVMTQSPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRADAAPTVSGGGGSGGGGSGGGGSVQLQQSGPELVKPGASVKISCKASGYSFTDYIMLWVKQSHGQSLEWIGNIDPYYGSTGYNLKFKGRATLTVDKSSSTAYMQLNSLTSEDSAVYYCARDGNYGSDYWGQGTTVTVSSGGGGSGGGGSKDEL

SEQ ID NO: 19 - DNA, 879 bp, synthetic construct
  source 1..879: mol_type = other DNA
gccaccatggccctccctgtcaccgccctgctgcttccgctggctcttctgctccacgccgctcggcccggatccggagatatcgtgatgacacaatcacctctttcacttcctgtgtcacttggagatcaagcatcaatctcatgcagatcatcacaatcaatcgtgcactcaaacggaaacacataccttgaatggtaccttcagaagcccggccaatcacctaaacttcttatctacaaagtgtcaaaccggtttagcggagtcccagataggttctctggatccggaagtggcaccgactttaccttgaagataagtagagtggaagcagaagatcttggagtttactactgctttcaaggatcacacgtgcctttcactttcggctctggcaccaaactagagatcaagcgcgctgatgcagcacctacagtgtcgggtggcggtggcagcggcggcggtggtagcggtggaggcggctcagtacagcttcaacaatcaggacctgaacttgtgaaacctggagcatcagtgaagatatcctgcaaagcatcaggatactcatttacagattacatcatgctttgggtgaaacaatcacacggacaatcacttgaatggatcggaaacatcgatccttactacggatcaacaggatacaaccttaaatttaaaggaagagcaacacttacagtggataaatcatcatcaacagcatacatgcaacttaactcacttacatcagaagattcagcagtctactactgcgcacgggatggaaactacggatcagattactggggccaggggacaaccgtcaccgtgagctccggcggcggggggagcgggggcgggggctccaaggacgagctgtga

SEQ ID NO: 20 - protein, 684 aa, synthetic construct
MALPVTALLLPLALLLHAARPGSGDIVMTQSPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPFTFGSGTKLEIKRADAAPTVSGGGGSGGGGSGGGGSVQLQQSGPELVKPGASVKISCKASGYSFTDYIMLWVKQSHGQSLEWIGNIDPYYGSTGYNLKFKGRATLTVDKSSSTAYMQLNSLTSEDSAVYYCARDGNYGSDYWGQGTTVTVSSASSGTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRLEGGGEGRGSLLTCGDVEENPGPRMGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 21 - DNA, 2061 bp, synthetic construct
  source 1..2061: mol_type = other DNA
gccaccatggccctccctgtcaccgccctgctgcttccgctggctcttctgctccacgccgctcggcccggatccggagatatcgtgatgacacaatcacctctttcacttcctgtgtcacttggagatcaagcatcaatctcatgcagatcatcacaatcaatcgtgcactcaaacggaaacacataccttgaatggtaccttcagaagcccggccaatcacctaaacttcttatctacaaagtgtcaaaccggtttagcggagtcccagataggttctctggatccggaagtggcaccgactttaccttgaagataagtagagtggaagcagaagatcttggagtttactactgctttcaaggatcacacgtgcctttcactttcggctctggcaccaaactagagatcaagcgcgctgatgcagcacctacagtgtcgggtggcggtggcagcggcggcggtggtagcggtggaggcggctcagtacagcttcaacaatcaggacctgaacttgtgaaacctggagcatcagtgaagatatcctgcaaagcatcaggatactcatttacagattacatcatgctttgggtgaaacaatcacacggacaatcacttgaatggatcggaaacatcgatccttactacggatcaacaggatacaaccttaaatttaaaggaagagcaacacttacagtggataaatcatcatcaacagcatacatgcaacttaactcacttacatcagaagattcagcagtctactactgcgcacgggatggaaactacggatcagattactggggccaggggacaacagtgacagtgtcatcagctagctccggaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcctcgagggcggcggagagggcagaggaagtcttctaacatgcggtgacgtggaggagaatcccggccctaggatgggaaccagcctcctctgttggatggccctgtgtctgctgggagcagatcacgcagacgcctgtccttacagcaacccaagcctctgcagcggaggaggaggaagcgaactgcctacacagggcaccttcagcaacgtgtccaccaacgtgtctccagccaagcctacaacaaccgcctgcccctacagcaacccaagcctgtgttccggaggaggaggatctccagctcctagacctcctacaccagcccctacaatcgcctctcagcctctgagcctgaggccagaggcctgcagaccagcagcaggaggagcagtgcacacaagaggcctggacttcgcttgcgacatctacatttgggctcctctggcaggaacttgtggagtcctgctgctgagcctggtcatcaccctctactgcaaccacaggaacaggagacgcgtctgcaagtgccctagacccgtggtctga

SEQ ID NO: 22 - DNA, 348 bp, synthetic construct
  source 1..348: mol_type = other DNA
gtacagcttcaacaatcaggacctgaacttgtgaaacctggagcatcagtgaagatatcctgcaaagcatcaggatactcatttacagattacatcatgctttgggtgaaacaatcacacggacaatcacttgaatggatcggaaacatcgatccttactacggatcaacaggatacaaccttaaatttaaaggaagagcaacacttacagtggataaatcatcatcaacagcatacatgcaacttaactcacttacatcagaagattcagcagtctactactgcgcacgggatggaaactacggatcagattactggggccaggggacaacagtgacagtgtcatca

SEQ ID NO: 23 - DNA, 363 bp, synthetic construct
  source 1..363: mol_type = other DNA
gatatcgtgatgacacaatcacctctttcacttcctgtgtcacttggagatcaagcatcaatctcatgcagatcatcacaatcaatcgtgcactcaaacggaaacacataccttgaatggtaccttcagaagcccggccaatcacctaaacttcttatctacaaagtgtcaaaccggtttagcggagtcccagataggttctctggatccggaagtggcaccgactttaccttgaagataagtagagtggaagcagaagatcttggagtttactactgctttcaaggatcacacgtgcctttcactttcggctctggcaccaaactagagatcaagcgcgctgatgcagcacctacagtgtcg

SEQ ID NO: 24 - protein, 6 aa, synthetic construct
GCCACC

SEQ ID NO: 25 - DNA, 45 bp, synthetic construct
  source 1..45: mol_type = other DNA
ggtggcggtggcagcggcggcggtggtagcggtggaggcggctca

SEQ ID NO: 26 - protein, 15 aa, synthetic construct
GGGGSGGGGSGGGGS

SEQ ID NO: 27 - DNA, 12 bp, synthetic construct
  source 1..12: mol_type = other DNA
aaggacgagctg

SEQ ID NO: 28 - protein, 4 aa, synthetic construct
KDEL

SEQ ID NO: 29 - DNA, 30 bp, synthetic construct
  source 1..30: mol_type = other DNA
ggcggcggggggagcgggggcgggggctcc

SEQ ID NO: 30 - protein, 10 aa, synthetic construct
GGGGSGGGGS

SEQ ID NO: 31 - DNA, 42 bp, synthetic construct
  source 1..42: mol_type = other DNA
ggcggcggggggagcgggggcgggggctccaaggacgagctg

SEQ ID NO: 32 - protein, 14 aa, synthetic construct
GGGGSGGGGSKDEL

SEQ ID NO: 33 - DNA, 30 bp, synthetic construct
  source 1..30: mol_type = other DNA
gagcagaaactgattagcgaagaggacctg

SEQ ID NO: 34 - protein, 10 aa, synthetic construct
EQKLISEEDL

SEQ ID NO: 35 - DNA, 63 bp, synthetic construct
  source 1..63: mol_type = other DNA
atggccctccctgtcaccgccctgctgcttccgctggctcttctgctccacgccgctcggccc

SEQ ID NO: 36 - protein, 21 aa, synthetic construct
MALPVTALLLPLALLLHAARP

SEQ ID NO: 37 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 38 - protein, 45 aa, synthetic construct
TTTPAPRPPTPAPTIASQPLSLRPEACRPAAGGAVHTRGLDFACD

SEQ ID NO: 39 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 40 - protein, 24 aa, synthetic construct
IYIWAPLAGTCGVLLLSLVITLYC

SEQ ID NO: 41 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 42 - protein, 42 aa, synthetic construct
KRGRKKLLYIFKQPFMRPVQTTQEEDGCSCRFPEEEEGGCEL

SEQ ID NO: 43 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 44 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 45 - DNA, 54 bp, synthetic construct
  source 1..54: mol_type = other DNA
gagggcagaggaagtcttctaacatgcggtgacgtggaggagaatcccggccct

SEQ ID NO: 46 - protein, 18 aa, synthetic construct
EGRGSLLTCGDVEENPGP

SEQ ID NO: 47 - protein, 157 aa, synthetic construct
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 48 - DNA, 474 bp, synthetic construct
  source 1..474: mol_type = other DNA
atgggaaccagcctcctctgttggatggccctgtgtctgctgggagcagatcacgcagacgcctgtccttacagcaacccaagcctctgcagcggaggaggaggaagcgaactgcctacacagggcaccttcagcaacgtgtccaccaacgtgtctccagccaagcctacaacaaccgcctgcccctacagcaacccaagcctgtgttccggaggaggaggatctccagctcctagacctcctacaccagcccctacaatcgcctctcagcctctgagcctgaggccagaggcctgcagaccagcagcaggaggagcagtgcacacaagaggcctggacttcgcttgcgacatctacatttgggctcctctggcaggaacttgtggagtcctgctgctgagcctggtcatcaccctctactgcaaccacaggaacaggagacgcgtctgcaagtgccctagacccgtggtctga

SEQ ID NO: 49 - DNA, 27 bp, synthetic construct
  source 1..27: mol_type = other DNA
tgtccttacagcaacccaagcctctgc

SEQ ID NO: 50 - protein, 9 aa, synthetic construct
CPYSNPSLC

SEQ ID NO: 51 - DNA, 720 bp, synthetic construct
  source 1..720: mol_type = other DNA
atggccggccccccccggctcctgctcctgcccctcctcctggctctcgcccggggcctgcccggcgctctcgccgcccaagaggtgcagcagtcccctcactgcaccaccgtgcccgtgggcgctagcgtgaacatcacctgcagcacaagcggcggcctgagaggcatctacctgagacagctgggccctcagccccaagacatcatctactacgaggacggcgtggtgcccaccaccgacagaagattcagaggcagaatcgacttcagcggcagccaagacaacctgaccatcaccatgcacagactgcagctgagcgacaccggcacctacacctgccaagccatcaccgaggtgaacgtgtacggcagcggcaccctggtgctggtgaccgaggagcagagccaaggctggcacagatgcagcgatgcccctcctcgggctagcgctctgcccgccccccctaccggctccgccctccctgatccccaaacagctagcgccctgcctgatcctcccgctgctagcgccctgcccgctgctctcgccgtgattagcttcctcctgggcctcgggctgggcgtggcctgcgtgctggctagaacacagatcaagaagctgtgcagctggagagacaagaacagcgccgcctgcgtggtgtacgaggacatgagccacagcagatgcaacaccctgagcagccccaatcagtatcag

SEQ ID NO: 52 - protein, 239 aa, synthetic construct
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 53 - DNA, 717 bp, synthetic construct
  source 1..717: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaag

(19) United States
(12) Patent Application Publication
(10) Pub. No.: US 2025/0041343 A1
(43) Pub. Date: Feb. 6, 2025

(54) CD7-CAR-T CELL, ITS PREPARATION METHOD AND THE APPLICATION THEREOF

(71) Applicants: Zhongwei XU, Beijing (CN); ADVANCED BIOTHERAPEUTICS, LTD, Suzhou (CN)

(72) Inventors: Zhongwei XU, Beijing (CN); Haiyan ZHANG, Beijing (CN)

(21) Appl. No.: 18/778,896

(22) Filed: Jul. 19, 2024

Related U.S. Application Data
(63) Continuation of application No. PCT/CN2023/070315, filed on Jan. 4, 2023.

(30) Foreign Application Priority Data
Feb. 28, 2022 (CN) 202210190015.3

(51) Int. Cl. A61K 35/17; A61K 39/00; C07K 14/705; C07K 14/725; C07K 16/28; C12N 5/0783; C12N 15/86

(52) U.S. Cl. CPC A61K 35/17; A61K 39/001111; A61K 39/4611; A61K 39/4631; C07K 14/7051; C07K 14/70517; C07K 16/2803; C12N 5/0636; C12N 15/86; A61K 2239/13; A61K 2239/17; A61K 2239/21; A61K 2239/25; C07K 2317/565; C07K 2317/622; C07K 2317/76; C07K 2319/02; C07K 2319/03; C12N 2740/15043

(57) ABSTRACT

The present invention discloses a CD7-CAR-T cell, its preparation method and the application thereof, wherein the CD7-CAR-T cell comprises an antibody targeting the CD7 antigen or its antigen-binding fragment and the antibody or its antigen-binding fragment contains a heavy chain variable region of the antigen complementary determining region CDR1, CDR2 and CDR3 with the amino acid sequence as shown in SEQ ID NO.: 12-14; and a light chain variable region of the antigen complementary determining regions CDR1, CDR2 and CDR3 with the amino acid sequence as shown in SEQ ID NO.: 15-17. The antibody and the CD7-CAR based on the antibody fragment of the present invention have a strong affinity with CD7 antigen molecules.

### CROSS REFERENCE TO RELATED APPLICATIONS

The present application claims the priority of CN 202210190015.3 submitted on Feb. 28, 2022, the entire content of which is incorporated herein by reference

### REFERENCE TO AN ELECTRONIC SEQUENCE LISTING

The contents of the electronic sequence listing (Sequence_listing.xml; Size: 56,316 bytes; and Date of Creation: Apr. 22, 2024) is herein incorporated by reference in its entirety.

### TECHNICAL FIELD

The present invention relates to the technical field of biological immunotherapy, specifically relates to a CD7-CAR-T cell, its preparation method and the application thereof.

### BACKGROUND ART

Acute lymphocytic leukemia (ALL) is a common malignant tumor in the blood system, mainly originating from the B and T lymphocyte lineages. Immature lymphocytes in the primitive form undergo multiple steps of specific gene damages to form tumor cells, which exhibit abnormal proliferation and aggregation in the bone marrow and inhibit the hematopoietic function of normal bone marrow stem cells, leading to the occurrence of ALL. Meanwhile, these tumor cells can also infiltrate extramedullary tissues (such as lymph nodes, meninges, liver, gonads, etc.), causing damages to related tissues.

ALL can occur in various age stages. The incidence rate of ALL in children and young people is relatively high, i.e. 3.6 in 100,000 and the incidence rate of ALL in adults is relatively low, i.e. 0.69 in 100,000. Initial chemotherapy and target therapy can kill invasive proliferating cells and selectively or acquired mutant cells, but drug insensitive ALL cells can still lead to disease recurrence. Bone marrow transplantation is an important treatment for ALL. Although allogeneic transplantation has good therapeutic effects on acute leukemia, most patients lack suitable bone marrow donors and are faced with the high cost of transplantation, making its application greatly limited. Autologous bone marrow transplantation as an alternative treatment to allogeneic transplantation has rapidly developed in recent years. Although a considerable amount of clinical data has been accumulated, the status of autologous bone marrow transplantation in the treatment of acute leukemia is still controversial due to the significant differences in efficacy reported. The long-term leukemia free survival rate (LFS) of autologous bone marrow transplantation during the initial complete remission period ranges from less than 30% to over 70%. Some data indicate that autologous bone marrow transplantation does not improve the LFS of patients with acute lymphoblastic leukemia, but there is also evidence that the efficacy of autologous bone marrow transplantation for acute lymphoblastic leukemia in complete remission is similar to that of allogeneic bone marrow transplantation, far superior to chemotherapy alone. Therefore, due to the uncertainty and low effectiveness of existing treatment methods, people need to continue exploring new and better treatment methods.

Acute T Cell Lymphocytic Leukemia (T-ALL) is a type of ALL, which is a malignant tumor caused by malignant transformation and clonal proliferation of T precursor cells in the bone marrow and thymus. The genetic mutations of T-ALL are diverse and highly heterogeneous, including gene deletions, mutations, chromosomal translocations, etc. These abnormalities can cause various signaling pathways (such as MAPK and Jak/Stat, PI3K/Akt/mTOR, etc.) and/or abnormalities in the cell cycle. Research has found that at least 170 potential oncogenic driver genes are associated with the occurrence of T-ALL.

T-ALL is more common in children and adolescents and can also occur in adults. T-ALL accounts for 10%-15% of pediatric ALL patients and approximately 25% of adult patients. Compared to acute B lymphoblastic leukemia (B-ALL), T-ALL is insensitive to chemotherapy, has poor efficacy, high recurrence rate, low remission rate and long-term survival rate. The advantage clones after recurrence is often rich in resistance genes to conventional chemotherapy drugs. Compared to pediatric T-ALL, the treatment effect of adult T-ALL is worse, with about 50% of adult patients relapsing about one year after treatment remission. The remission rate of rechemotherapy is only 30%-45% and only 40% of patients can achieve long term survival. Moreover, the prognosis of relapsed/refractory T-ALL patients is usually worse.

In recent years, chimeric antigen receptor T cell (CAR-T) therapy has shown significant clinical efficacy in the treatment of hematological tumors, bringing new hope to tumor treatment. CAR-T therapy targeting CD19 can achieve a CR of 90%-100% in B-ALL and both autologous and allogeneic CAR-T treatments can achieve a recurrence free survival state of more than 5 years. One of the key technologies of CAR-T therapy is to select a specific target site expressed on tumor cells, such as tumor associated antigen or tumor specific antigen and to prepare CAR-T cells based on the specific antibody of this antigen for treatment.

CD7 is a cell membrane single chain glycoprotein with a molecular weight of 40 kDa, mainly expressed in thymocytes and most peripheral blood T lymphocytes and appears before other T lineage antigens (CD1, CD2, CD3, CD4, CD5, CD8) and also before TCR-β chain gene rearrangement. CD7 antigen is also expressed in NK cells and part of myeloid cells and is highly expressed on the surface of leukemia cells originating from the T lymphoid system. In addition to T-ALL, the vast majority of T-cell lymphoma, NK and NKT lymphoma also express CD7, with a 20-30% expression rate in acute myeloid leukemia (AML). However, animal experiments have shown that mouse T progenitor cells that have disrupted CD7 molecules still produce normal T cell development and in vivo balance, with little change in T cell effector function, suggesting that CD7 does not have a critical impact on T cell development and function. Therefore, CD7 is an ideal target for T-ALL and CD7 positive tumors.

Compared to CAR-T therapy for B-ALL, the application of CD7-CAR-T cell targeted therapy for T cell acute lymphoblastic leukemia and CD7 positive lymphoma still faces significant technical challenges. This is because normal effector T cells and T cell tumors both express CD7 antigen, which can lead to the “fratricide” effect of CD7-CAR-T cells. Therefore, it is difficult to successfully prepare CD7-CAR-T cells in vitro; in clinical applications, it is also important to consider not allowing CD7-CAR-T cells to exist indefinitely in the body so as to avoid serious side effects. At present, there are only a few studies on CD7-CAR-T technology. In order to avoid the “fratricide” effect, CRISPR gene editing is used to knock out the CD7 gene in T cells. However, it is difficult to completely knock out the CD7 molecule in practice and theory and there is a risk of clinical application of graft-versus-host disease (GVHD). Meanwhile, the natural “off target effect” of the gene editing may also limit the widespread clinical application due to potential side effects. The application of nano body technology to reduce the volume of antibody binding regions has certain advantages, but the disadvantage can be that the shrunken single arm antibodies can lead to a relative decrease in the binding force and stability of the antigen and the antibody, resulting in a decrease in the efficacy of CD7-CAR-T cells. In addition, normal T cells, NK cells, etc. also have CD7 expression, which is an important force in maintaining normal cellular immunity, fighting against viral and bacterial infections in the body. It only emphasizes the long-lasting killing effect of CD7-CAR-T in the body, which can cause long-term infections and other side effects while eliminating tumors. Therefore, reasonable control of the existence of CD7-CAR-T cells is also an important factor in excellent CAR-T design.

The information in the background art is only intended to illustrate the overall background of the present invention and should not be regarded as acknowledging or implying in any form that this information constitutes the prior art known to those skilled in the art.

### SUMMARY OF THE INVENTION

To solve the technical problems in the prior art, the present inventor conducted in-depth researches, prepared and selected a unique anti-human CD7 monoclonal antibody sequence as the antigen recognition element constructed by CD7-CAR-T of the present invention. Meanwhile, the structure where the antibody fragment against the CD7 antigen is fused with the endoplasmic reticulum (ER) localization signal is applied to localize the CD7 antigen molecule in the ER reticulum of T cells, blocking its expression on the surface of CAR-T cells, thereby eliminating the “fratricide” effect of CD7-CAR-T cells and facilitating the in vitro preparation and production of CD7-CAR-T cells. In addition, the present invention applies a suicide gene structure to CAR-T structures, which can eliminate CAR-T cells when they are not needed to ensure the safety of its application. Specifically, the present invention includes the following content.

The first aspect of the present invention provides an antibody or the antigen binding fragment thereof, comprising a heavy chain variable region of antigen complementary determining regions CDR1, CDR2 and CDR3 with an amino acid sequence as shown in SEQ ID NO.: 12-14; and
the light chain variable region of the antigen complementary determining regions CDR1, CDR2 and CDR3 with an amino acid sequence as shown in SEQ ID NO.: 15-17.

According to the antibody or the antigen binding fragment thereof of the present invention, preferably, the antibody has any one of the amino acid sequences as shown in (I), (II) or (III):
(I) a heavy chain variable region amino acid sequence as shown in SEQ ID NO.: 9 and a light chain variable region amino acid sequence as shown in SEQ ID NO.: 11;
(II) An amino acid sequence with at least 90%, preferably at least 95%, more preferably at least 98% and most preferably at least 99% homology to the amino acid sequences as shown in SEQ ID NO.: 9 and 11;
(III) An amino acid sequence obtained by subjecting the amino acid sequences as shown in SEQ ID NO.: 9 and 11 to modification, substitution, deletion or addition of one or more amino acids;
wherein, the amino acid sequence has the activity of antibodies against CD7 antigen.

Preferably, according to the antibody or the antigen binding fragment thereof of the present invention, the antibody comprises at least one of a polyclonal antibody, a monoclonal antibody, a chimeric antibody, a humanized antibody or a bispecific antibody; the antigen binding fragments include at least one of a Fab fragment, a Fab′, a F(ab′)2 fragment, a single chain variable fragment scFv, a scFv-Fc fragment or a single chain antibody ScAb.

The second aspect of the present invention provides a CD7 blocking molecule comprising:
a. the antibody or the antigen binding fragment thereof according to the first aspect; and
b. an endoplasmic reticulum localization domain.

A third aspect of the present invention provides a chimeric antigen receptor, comprising:
1) an antigen binding domain recognizing the CD7 antigen, wherein the antigen binding domain comprises the antibody or the antigen binding fragment thereof according to the first aspect;
2) a transmembrane structural domain; and
3) an intracellular signal transduction domain;
Preferably, the chimeric antigen receptor further comprises a hinge area;
Preferably, the chimeric antigen receptor further comprises a suicide switch molecule;
Preferably, the chimeric antigen receptor further comprises an intracellular costimulatory domains;
Preferably, the transmembrane domain is selected from at least one peptides of CD28, NKp30, CDS, DAP10, 4-1BB, DAP12, CD3C, CD3E, CD45, CD4, CD5, CD8, CD9, CD16, CD22, CD33, CD37, CD64, CD80, CD86, CD134, CD137, CD154, KIRDS2, OX40, CD2, CD27, LFA-1, ICOS (CD278), 4-1BB (CD137), GITR, CD40, BAFFR, HVEM (LIGHT), SLAMF7, NKp80 (KLRF1), CD160, CD19, IL2R β, IL2Rγ, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96, CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp or any combination thereof;
Preferably, the intracellular signal transduction domain is selected from at least one of CD8, CD3ζ, CD3δ, CD3γ, CD3ε, FcγRI-γ, FcγRIII-γ, FcεRIβ, FcεRIγ, DAP10, DAP12, CD32, CD79a, CD79b, CD28, CD3C, CD4, b2c, CD137 (4-1BB), ICOS, CD27, CD288, CD80, NKp30, OX40 or any combination thereof.

The fourth aspect of the present invention provides a separated nucleic acid molecule encoding the antibody or the antigen binding fragment thereof according to the first aspect of the present invention or the blocking molecule according to the second aspect or the chimeric antigen receptor according to the third aspect.

The fifth aspect of the present invention provides a carrier comprising the nucleic acid molecule according to the fourth aspect.

The sixth aspect of the present invention provides a host cell comprising the carrier according to the fifth aspect.

A seventh aspect of the present invention provides a preparation method for the chimeric antigen receptor according to the third aspect, comprising culturing host cells according to the sixth aspect.

An eighth aspect of the present invention provides an immunologic effector cell that expresses the antibody or the antigen binding fragment thereof according to the first aspect of the present invention, the blocking molecule according to the second aspect or the chimeric antigen receptor according to the third aspect;

Preferably, the immunologic effector cells are selected from at least one of a white blood cell, a monocyte, a macrophage, a dendritic cell, a mast cell, a neutrophil, a basophil, an eosinophil, an αβ T cell, a γδT cell, a natural killer (NK) cell, a natural killer T (NKT) cell, a B cell, a natural lymphoid like cell (ILC), a cytokine induced killer (CIK) cell, a cytotoxic T lymphocyte (CTL), a lymphokine activated killer (LAK) cell, a T lymphocyte, a peripheral blood mononuclear cell and a hematopoietic stem cell.

The ninth aspect of the present invention provides an application of reagents in the preparation of compositions, drugs, preparations or test kits for the prevention and/or treatment of cancer or tumors, wherein the reagents include the antibody or the antigen binding fragments thereof according to the first aspect of the present invention or the blocking molecule according to the second aspect or the chimeric antigen receptor according to the third aspect or the immunologic effector cell according to the eighth aspect.

Preferably, the cancer or tumor refers to a cancer or tumor related to CD7 expression, further preferably, the cancer or tumor is a hematological malignancy; further preferably, the hematological malignancy is a T-cell related tumor, which includes leukemia, lymphoma and myeloma.

The tenth aspect of the present invention provides an application of the antibody or the antigen binding fragment thereof according to the first aspect of the present invention or the blocking molecule according to the second aspect or the chimeric antigen receptor according to the third aspect or the immunologic effector cell according to the eighth aspect in combination administration with other drugs. Other medications include but are not limited to diagnostic agents, prophylactic agents and/or therapeutic agents.

The excellent technical effects of the present invention include but are not limited to the following facts. The antibody and the CD7-CAR based on the antibody fragment of the present invention have extremely strong affinity with CD7 antigen molecules and the blocking molecule of the present invention can almost completely block the expression of CD7 molecules on the cell surface without affecting the normal proliferation of T cells, thereby effectively avoiding the fratricide of CD7-CAR-T cells. In addition, the CD7-CAR-T cells of the present invention have a significant and specific killing effect on CD7 positive target cells, providing beneficial CAR-T cells for clinical application of the cell therapy. Meanwhile, the present invention applies suicide gene structures to CAR-T structures, which can eliminate CAR-T cells when they are not needed so as to ensure the safety of its application.

### BRIEF ILLUSTRATION OF DRAWINGS

FIG. 1 shows the plasmid map of the third-generation lentivirus vector pCDH-EF1(X6)-MCS-T2A-Puro.

FIG. 2 is a schematic diagram of the CD7-Blocker molecular structure.

FIG. 3 is a schematic diagram of the CD7-CAR molecular structure.

FIG. 4 shows the flow cytometry analysis for the blocking effect of CD7-Blocker against the CD7 molecules on the Jurkat cell surface, wherein, A to E correspond to the expression of CD7 molecules on the Jurkat cell surface transduced by different viral MOI values, while M0, M2.5, M5, M10 and M15 represent a MOI value of 0, 2.5, 5, 10 and 15, respectively.

FIG. 5 shows the flow cytometry analysis of the blocking effect of CD7-Blocker against CD7 molecules on the T cell surface.

FIG. 6 shows the flow cytometry detection of CD7 molecule positivity and CAR positivity on the T cell surface.

FIG. 7 shows the amplification curve of the CD7-Blocker-CAR-T cell.

FIG. 8 shows the killing curve of the CD7-Blocker-CAR-T cell cocultured with the positive target cell U87-CD7-eGFP, wherein the cocultured cell groups corresponding to the three curves are the individual target cell U87-CD7-eGFP (a), the control CD7-Blocker-T: U87-CD7-eGFP (b) and CD7-Blocker-CAR-T: U87-CD7-eGFP (c).

FIG. 9 shows the killing curve of the CD7-Blocker-CAR-T cell cocultured with the negative target cell U87, wherein the cocultured cell groups corresponding to the three curves are the control target cell U87 (b′), the control CD7-Blocker-T: U87 (a′) and CD7-Blocker-CAR-T: U87 (c′), respectively.

FIG. 10 shows the killing efficiency of the CD7-Blocker-CAR-T on the CD7 positive target cell U87-CD7-eGFP.

### SPECIFIC EMBODIMENTS

A detailed description of various exemplary embodiments of the present invention are provided, which should not be construed as a limitation of the present invention, but rather as a more detailed description of certain aspects, features and embodiments of the present invention. If specific technology or conditions are not specified in the embodiment, the technologies or conditions described in the literature within the art (such as referring to Molecular Cloning Experiment Guidelines by J. Sambrook et al., translated by Huang Peitang et al., third edition by Science Press) prevail or are conducted following the product manual. The used reagents or instruments without specifying the manufacturer are conventional products that can be obtained through market procurement.

It should be understood that the terms described in the present invention are only for describing specific embodiments and are not intended to limit the present invention. Furthermore, for the numerical range in the present invention, it should be understood as specifically disclosing the upper and lower limits of the range, as well as each intermediate value between them. The intermediate value within any stated values or stated range and any other stated value or each smaller range between intermediate values within the stated range is also included in the present invention. The upper and lower limits of these smaller ranges can be independently included or excluded within the range.

Unless otherwise specified, all technical and scientific terms used in present invention have the same meanings as those commonly understood by those skilled in the art. Although the present invention only describes preferred methods and materials, any methods and materials similar or equivalent to those described herein may also be used in the embodiment or testing of the present invention. All literature mentioned in present description is incorporated by reference to publicly disclose and describe methods and/or materials related to the literature. In case of conflict with any incorporated literature, the content of present description shall prevail.

The heavy chain variable region and light chain variable region of antibodies typically comprise three complementary determining regions (CDR) and four skeletal regions (FR). Complementary determining regions are connected by skeleton regions and when recognizing antibodies, FR molecules curl up to bring CDR molecules closer to each other. The complementary determining region is the binding site where the antibody or the antigen binding fragment thereof binds with the antigen, which means the sequence of the complementary determining region determines the specificity of the antibody. As understood in the art, the antibody is a glycoprotein that contains at least two heavy (H) chains and two light (L) chains interconnected by a disulfide bond or the antigen-binding portion thereof. The heavy chain includes the variable heavy chain region (VH) and the constant heavy chain region (CH). The light chain includes a light chain variable region (VL) and a light chain constant region (CL). The variable regions of the heavy chain and the light chain include the frame region (FR) and the complementary decision region (CDR). The four FRs are relatively conservative while the CDR regions (CDR1, CDR2 and CDR3) contain highly variable regions.

The “antigen binding fragment” in present invention refers to a polypeptide fragment that contains a portion of the intact antibody, such as the antigen binding region or the variable region of the intact antibody, and has the characteristic of specifically targeting CD7. Preferably, it contains at least one CDR in the antibody heavy chain variable region and/or the light chain variable region; more preferably, it may contain CDR1-3 in a heavy chain variable region and/or CDR1-3 in a light chain variable region. The antigen binding fragment can be prepared through various techniques, including but not limited to hydrolysis and digestion of intact antibody proteins or production by expression of host cells containing antigen binding fragments.

The present invention provides the antibody or the antigen binding fragment targeting CD7, which have good safety and targeting properties and can specifically bind to the extracellular domain of human CD7. By the application of the vector containing the encoding sequence of the antibody or the antigen binding fragment thereof in infecting immune cells, immunologic effector cells with significant killing ability against CD7 expressing tumor cells can be obtained. These immunologic effector cells can be applied to treat or improve CD7 expression related diseases, laying the foundation for the treatment of CD7 positive tumors.

Without limitations or theoretical constraints, the sequences of heavy chain variable regions CDR1, CDR2, CDR3 and light chain variable regions CDR1, CDR2 and CDR3 of the antibody or the antigen binding fragment thereof can be randomly selected within the following range: the heavy chain variable regions of antigen complementary determining regions CDR1, CDR2 and CDR3 with amino acid sequences as shown in SEQ ID NO.: 12-14; the light chain variable region of the antigen complementary determining regions CDR1, CDR2 and CDR3 with the amino acid sequence as shown in SEQ ID NO.: 15-17.

In the present invention, the antibody or the antigen binding fragment thereof has any one of the amino acid sequences as shown in (I), (II) or (III): a heavy chain variable region amino acid sequence as shown in SEQ ID NO.: 9 and a light chain variable region amino acid sequence as shown in SEQ ID NO.: 11; (II) an amino acid sequences with at least 90%, preferably at least 95%, more preferably at least 98% and most preferably at least 99% homology to the amino acid sequences as shown in SEQ ID NO.: 9 and 11; (III) an amino acid sequences obtained by subjecting the amino acid sequences as shown in SEQ ID NO.: 9 and 11 to the modification, substitution, deletion or addition of one or more amino acids. It should be noted that the homology (sometimes referred to as “identity” in present invention) sequence mentioned above does not alter the antigen antibody binding characteristics, which means the selected amino acid sequences still retain the activity of antibodies against the tumor surface antigen CD7.

Preferably, the encoding sequence of the heavy chain variable region is shown in SEQ ID NO.: 22 and the encoding sequence of the light chain variable region is shown in SEQ ID NO.: 23.

Preferably, the above-mentioned amino acid sequence in the present invention is one obtained by subjecting the coding sequence of mouse derived antibodies to the host codon preference and then expression. In the present invention, the host codon preference modification refers to the base substitution on base sequences based on degenerate codons to meet the needs of different host expressions. The codon preference modification generally does not change the sequence of product proteins or peptides. In the coding sequence of the mouse antibody, the coding sequence of the heavy chain variable region is shown as SEQ ID NO.: 8 and the coding sequence of the light chain variable region is shown as SEQ ID NO.: 10.

Preferably, the antibody comprises at least one of a monoclonal antibody, a humanized antibody, a chimeric antibody and a bispecific antibody; the antigen binding fragments are at least one of Fab, F(ab′), F(ab′)2, Fd, a single chain antibody scFv, a disulfide linked Fv(sdFv) or a single domain antibody. More preferably, the antibody or the antigen binding fragment thereof is humanized.

Preferably, the antibody further comprises an antibody constant region; preferably, the antibody constant region is selected from the constant region of any one of IgG1, IgG2, IgG3, IgG4, IgA, IgM, IgE and IgD.

Preferably, the heavy chain constant region of the antibody constant region is selected from the heavy chain constant region of any one of IgG1, IgG2, IgG3 or IgG4, preferably the heavy chain constant region of IgG4; the light chain constant region of the antibody constant region is κ or λ.

The antibody of the present invention may comprise an Fc region, which is derived from IgG, such as IgG1, IgG2, IgG3 or IgG4.

The term “monoclonal antibody” used in present invention, sometimes also known as “monoclonal antibody” or mAb, refers to an immunoglobulin obtained from a pure cell line, which has the same structure and chemical properties and is specific to a single antigenic determinant. Monoclonal antibodies are different from conventional polyclonal antibody preparations (usually with different antibodies targeting different determinant clusters), as each monoclonal antibody targets a single determinant cluster on an antigen. In addition to their specificity, the advantage of monoclonal antibodies is that they are obtained through hybridoma or recombinant engineering cell culture and do not mix with other immunoglobulins. The modifier “monoclonal” indicates the characteristics of an antibody obtained from a homogeneous antibody population, but this should not be interpreted as requiring any special or specific method to produce the antibody.

Variant antibodies are also included within the scope of the present invention. The present invention does not specifically limit the variant sequence, as long as they have binding properties with targeting CD7 antigens or antibodies with increased affinity. Other variants with such sequences can be obtained using methods known in the art and are all included within the scope of the present invention. Those skilled in the art can modify the amino acid sequence of peptides by utilizing recombination methods and/or synthetic chemistry techniques for producing variant polypeptides. For example, the amino acid substitution can be used to obtain antibodies with further enhanced affinity. Optionally, the codon optimization of nucleotide sequences can be used to improve translation efficiency in expression systems used for antibody production. Such variant antibody sequences have sequence identity of 80% or higher (i.e. 85%, 90%, 95%, 96%, 97%, 98%, 99% or greater) with the sequences listed in the present invention. The sequence identity is calculated in relative to the sequences listed in the present invention. The best comparison is performed through such as the program GAP or using BESTFIT with default gap weights.

The term “modification” used in present invention refers to the fact that the amino acid modification will not significantly affect or alter the binding characteristics of antibodies containing that amino acid sequence. This type of modification includes substitution, addition and deletion of amino acids. Preferably, different residue positions vary due to the substitution of conserved amino acids. The antibodies of the present invention may include glycosylation, acetylation, phosphorylation, amidation, derivatization through known protective/blocking groups, protein hydrolysis cleavage or non naturally occurring amino acid modifications.

Conservative amino acid substitution refers to the interchangeability of residues with side chains. For example, the amino acid groups with aliphatic side chains are glycine, alanine, valine, leucine and isoleucine; the amino acid groups with aliphatic hydroxyl side chains are serine and threonine; the amino acid groups with amide side chains are asparagine and glutamine; the amino acid groups with aromatic side chains are phenylalanine, tyrosine and tryptophan; the amino acid groups with alkaline side chains are lysine, arginine and histidine; the amino acid groups with sulfur-containing side chains are cysteine and methionine. The preferred conservative amino acid substitution groups are: valine leucine-isoleucine, phenylalanine-tyrosine, lysine-arginine, alanine-valine, glutamic acid-aspartic acid and asparagine-glutamine. Therefore, one or more amino acid residues in the CDR region of the antibody of the present invention can be replaced with other amino acid residues from the same side chain family.

Another possible type of variable region modification is the mutation of amino acid residues in the CDR1, CDR2 and/or CDR3 regions of VH and/or VL to improve one or more binding properties of the target antibody (such as affinity). Mutations can be introduced through targeted mutagenesis or PCR transduced mutagenesis. Preferably the conservative modification as described above is introduced. Mutations can be substitutions, additions or deletions of amino acids, but substitution is preferred. In addition, the variation of residues in the CDR region usually does not exceed one, two, three, four or five.

The present invention provides a CD7 blocking molecule comprising an antibody or the antigen binding fragment thereof and an endoplasmic reticulum localization domain according to the present invention. Preferably, it has an amino acid sequence as shown in SEQ ID NO.: 18 and its coding sequence is as shown in SEQ ID NO.: 19. Preferably, the endoplasmic reticulum localization domain has an amino acid sequence as shown in SEQ ID NO.: 32, with the encoding sequence as shown in SEQ ID NO.: 31. The endoplasmic reticulum localization domain contains ER localization signal molecules, which have an amino acid sequence as shown in SEQ ID NO.: 28 and an encoding sequence as shown in SEQ ID NO.: 27.

The blocking molecule of the present invention can locate the antibody fragment of the present invention into the ER reticulum and the CD7 antigen molecule is intercepted by the antibody fragment in the ER reticulum in the secretion pathway, so it cannot be properly located on the cell surface, thus avoiding the “fratricide” between CD7-CAR-T cells. In another embodiment, the linker connecting the antibody fragment of the present invention to the ER localization signal molecule is a myc sequence, which has the amino acid sequence as shown in SEQ ID NO.: 33.

The present invention also provides an anti-human CD7 chimeric antigen receptor CAR comprising an antigen binding domain (sometimes referred to as an “antigen recognition region” in present invention) that recognizes CD7 antigens, a hinge region, a transmembrane domain (sometimes referred to as a “transmembrane region” in present invention) and an intracellular signaling domain (sometimes referred to as an “intracellular region” in present invention), wherein the antigen recognition region comprises an antibody or antigen-binding fragment specifically binding to CD7 as described in the present invention.

In the absence of limitations, the antigen recognition region can be monovalent or multivalent (such as divalent or trivalent). The antigen binding region can be single specific or multi-specific (such as bispecific). Bispecific can be targeting CD7 and another antigen or it can be targeting two different epitopes of CD7. Preferably, the antigen recognition region is a single chain antibody (monovalent or multivalent). Single chain antibody scFv includes a heavy chain variable region and a light chain variable region. The heavy chain variable region and the light chain variable region are connected by linkers to form antibodies.

Preferably, the connection method for scFv heavy and light chains is VH-Linker-VL or VL-Linker-VH. In some embodiments, the sequence of Linker can use the existing linker sequences. Preferably, the sequence of Linker is (GGGGS) n, where n=1-6. Further preferably, the sequence of Linker is GGGGSGGGGGGGS.

Preferably, the CAR further comprises a leading signal peptide sequence. Generally speaking, signal peptides are peptide sequences that enable polypeptides to target the desired site in cells. In some embodiments, signal peptides allow the polypeptides to target the secretion pathways of cells and will allow the polypeptides to be integrated and anchored into the lipid bilayer. In some embodiments, the signal peptide is a membrane localization signal peptide. Preferably, the leading peptide sequence is derived from the leading peptide sequence of CD8; more preferably, the CD8 leading peptide sequence has an amino acid sequence as shown in SEQ ID NO.: 36.

The “hinge region”, “transmembrane region” and “intracellular region” in present invention can all be selected from the sequences of hinge regions, transmembrane regions and intracellular regions in existing known CAR-T techniques.

The hinge region of the chimeric antigen receptor is located between the extracellular antigen binding region and the transmembrane region. The hinge region is an amino acid segment that typically exists between two domains of a protein and allows for protein flexibility and the relative movement between the two domains. The hinge region can be the hinge region of a naturally occurring protein or a part thereof. The hinge region of antibodies (such as IgG, IgA, IgM, IgE or IgD antibodies) can also be used for chimeric antigen receptors as described in present invention. Non naturally occurring peptides can also be used as the hinge region of the chimeric antigen receptor described in present invention. In some embodiments, the hinge region is a peptide linker. Preferably, the hinge area originates from CD8α. Preferably, the hinge region of the CD8α has an amino acid sequence as shown in SEQ ID NO.: 38.

The transmembrane region of chimeric antibody receptors can form an a Spiral, a complexes with more than one α Spiral, βBarrel or any other stable structure that can cross the domain cell phospholipid bilayer. The transmembrane region can be of natural or synthetic origin. The transmembrane region can originate from CD3ε, CD4, CD5, CD8α, CD9, CD16, CD22, CD28, CD33, CD37, CD45, CD64, CD80, CD86, CD134, CD137, CD154, α, β or ζ chains of a T cell receptor. Preferably, the transmembrane region originates from CD8α. Preferably, the transmembrane region of the CD8α has an amino acid sequence as shown in SEQ ID NO.: 40.

Preferably, the intracellular region of the chimeric antigen receptor comprises a signal transduction region and/or a costimulatory signal transduction region. The number of signal transduction areas and/or costimulatory signal transduction areas can both be one or more.

The intracellular signaling pathway is responsible for the activation of at least one normal effector function in immunologic effector cells that express chimeric antigen receptors. For example, the effector function of T cells can be cell lysis activity or helper activity, including cytokine secretion. Although the entire intracellular signal transduction region can usually be utilized, in many cases, the use of the entire chain is unnecessary. As for using the truncated portion of the intracellular signal transduction region, as long as its transduction effector functional signal is used, this truncated portion can be used instead of the complete chain. Therefore, the intracellular signal transduction region comprises any truncated form of the intracellular signal transduction region that is sufficient to transduce effector functional signals. In some embodiments, the signal transduction area originates from at least one of CD3ζ, FcRγ(FCER1G), FcRβ(Fc)εRib), CD3γ, CD3δ, CD3ε, CD5, CD22, CD137, CD79a, CD79b and CD66d. Preferably, the intracellular region originates from human CD33 intracellular region. Furthermore, the human CD3ζ intracellular region has an amino acid sequence as shown in SEQ ID NO.: 44.

In addition to the stimulation of antigen-specific signals, many immunologic effector cells also require co-stimulation to promote cell proliferation, differentiation and survival, as well as to activate the effector function of cells. The “costimulatory signal transduction region” can be the cytoplasmic portion of costimulatory molecules. The term “costimulatory molecule” refers to an associated binding partner on immune cells (such as T cells) that specifically binds to costimulatory ligands, thereby mediating costimulatory responses through immune cells, such as but not limited to proliferation and survival. The costimulatory signal transduction area can originate from the intracellular signaling region of at least one of CARD11, CD2, CD7, CD27, CD28, CD30, CD40, CD54, CD83, OX40, CD137, CD134, CD150, CD152, CD223, CD270, PD-L2, PD-L1, CD278, DAP10, LAT, NKD2C, SLP76, TRIM, FcεRIγ, MyD88 and 4-1BB. In some embodiments, the costimulatory signal transduction area originates from 4-1BB. In some embodiments, the 4-1BB costimulatory signal transduction region comprises the amino acid sequence as shown in SEQ ID NO.: 42.

Preferably, the nucleotide sequence of the CAR is shown as SEQ ID NO.: 20 and its coding sequence is shown as SEQ ID NO.: 21.

In order to address the various toxic side effects associated with CAR-T cell therapy and increase the safety of CAR-T cell therapy, the chimeric antigen receptor CAR designed by the inventor further includes the “suicide switch” a RQR8 molecule, which has an amino acid sequence as shown in SEQ ID NO.: 47 and a coding sequence as shown in SEQ ID NO.: 48. The RQR8 molecule is fused with the intracellular signaling domain CD3ζ in the CD7-CAR structure by a T2A binding peptide with an auto cleavage function. Preferably, the RQR8 molecule carries two CD20 antigenic epitopes and can induce T cell apoptosis by targeting CD20 by using the anti-CD20 rituximab and activating the antibody dependent cell-transduced cytotoxicity (ADCC) and the complement transduced cytotoxicity (CDC). When necessary, the use of drugs such as rituximab can achieve the elimination of CAR-T cells, thereby increasing the safety of CAR-T cell therapy.

The present invention provides a separated nucleic acid that encodes the antibody or the antigen binding fragment thereof or the blocking molecule or a chimeric antigen receptor as described earlier.

The present invention provides a carrier comprising the isolated nucleic acid as described in the present invention. The vector can be an expression vector or a cloning vector. In some embodiments, the vector is a viral vector. Virus vectors include but are not limited to an adenovirus vector, an adeno-associated virus vector, a lentivirus vector, a retrovirus vector, a cowpox vector, a herpes simplex virus vector and their derivatives.

The present invention provides a host cell comprising the aforementioned carrier. The suitable host cells for cloning or expressing DNA are prokaryotic cells, yeast cells or higher eukaryotic cells. Common examples of prokaryotic host cells include Escherichia coli, Bacillus subtilis, etc. The commonly used eukaryotic host cells include a yeast cell, an insect cell, a mammalian cell, etc.

The present invention provides a preparation method for an anti-human CD7 chimeric antigen receptor CAR, which includes culturing the host cells mentioned above. Preferably, the culture conditions of the preparation method are sufficient to enable host cells to the anti-human CD7 express chimeric antigen receptor CAR.

The present invention provides an immunologic effector cell that expresses a specific CD7 binding antibody or the antigen binding fragment thereof or a blocking molecule or an anti-human CD7 chimeric antigen receptor CAR.

In the present invention, “immunologic effector cells” are immune cells that can perform immune effector functions. In some embodiments, immunologic effector cells express at least FcγRIII and execute ADCC effector functions. Examples of immunologic effector cells that transduce ADCC include a peripheral blood mononuclear cell (PBMC), a natural killer (NK) cell, a monocyte, a cytotoxic T cell, a neutrophil and an eosinophil. Preferably, the immunologic effector cells are selected from at least one of an immune cell, a T lymphocyte, a NK cell, a peripheral blood mononuclear cell (PBMC) and a hematopoietic stem cell cultured and differentiated from pluripotent stem cells or embryonic stem cells. More preferably, the immunologic effector cells are T lymphocytes (same to T cells). In some embodiments, T cells can be CD4+/CD8−, CD4−/CD8+, CD4+/CD8+, CD4−/CD8− or their combination. In some embodiments, T cells produce IL-2, IFN and/or TNF when expressing chimeric antigen receptors and binding to target cells. In some embodiments, CD8+ T cells lyse antigen-specific target cells when expressing chimeric antigen receptors and binding to target cells.

The present invention provides a method for preparing immunologic effector cells, which comprises infecting immunologic effector cells with isolated nucleic acids or carriers as described in the present invention. Preferably, the present invention prepares genetically modified immunologic effector cells by introducing chimeric antigen receptors into immunologic effector cells (such as T cells).

It should be noted that the method of introducing nucleic acids or vectors into mammalian cells is known in the art and the vectors can be transferred into immunologic effector cells through physical, chemical or biological methods. The physical methods used to introduce carriers into immunologic effector cells include the calcium phosphate precipitation, the liposome transfection, the particle bombardment, the microinjection, the electroporation and so on. Chemical means for introducing nucleic acids or carriers into immunologic effector cells include a colloidal dispersion system, such as macromolecular complexes, nanocapsules, microspheres, beads and lipid based systems (including oil in water lotion, micelles, mixed micelles and liposomes). An exemplary colloidal system used as an in vitro delivery medium is liposomes (such as artificial membrane vesicles). Biological methods for introducing nucleic acids or vectors into immunologic effector cells include the use of DNA and RNA vectors. Virus vectors have become the most widely used method for inserting genes into mammalian cells, such as human cells. In some embodiments, transfected or transfected immunologic effector cells proliferate in vitro after introducing nucleic acids or vectors.

In some embodiments, the preparation also includes further evaluation or screening of transfected or transfected immunologic effector cells to select modified immunologic effector cells.

The present invention further provides a drug or drug composition comprising at least one of an antibody specifically binding to CD7 or the antigen-binding fragment thereof, a separated nucleic acid, a carrier, a chimeric antigen receptor CAR, a separated nucleic acid, an anti-human CD7 chimeric antigen receptor CAR prepared by a method for preparing the chimeric antigen receptor CAR, an immunologic effector cell and immunologic effector cells obtained by a method for preparing the immunologic effector cell.

In some embodiments, the drug composition further includes a pharmaceutically acceptable carrier.

The drug compositions can be prepared in the form of freeze-dried formulations or aqueous solutions by mixing active agents with optional pharmaceutically acceptable carriers of desired purity. Pharmaceutically acceptable carriers are non-toxic to the recipient at the used dosage and concentration and may include at least one of a buffering agent, an antioxidant, a preservative, an isotope, a stabilizer and a surfactant. In addition, in order for drug compositions to be suitable for in vivo administration, they must be sterile. The drug composition can be made sterile by filtering through a sterile filtration membrane.

In some embodiments, the drug composition may contain at least one additives of a cytotoxic agent, a chemotherapeutic agent, a cytokine, an immunosuppressant, a growth inhibitor and an active agent required for specific indications to be treated. The specific amount of additives can be adjusted according to actual needs.

The present invention also provides the application of reagents in the preparation of drugs or drug compositions for treating or improving cancer, wherein the reagents are selected from at least one of the antibody specifically binding to CD7 or the antigen-binding fragment thereof, an isolated nucleic acid, a carriers, a host cell, an anti-human CD7 chimeric antigen receptor CAR, anti-human CD7 chimeric antigen receptor CARs obtained by the preparation method for the immunologic effector cell, an immunologic effector cell and an immunologic effector cell.

Preferably, the treatment or improvement of cancer refers to the ability to stimulate or enhance the immune function of cancer patients.

Preferably, the cancer refers to cancer associated with CD7 expression.

In present invention, “cancer related to CD7 expression” refers to diseases directly or indirectly caused by abnormal CD7 expression, usually referring to diseases caused by CD7 overexpression. Preferably, the cancer or tumor is a hematological malignancy. Further preferably, the hematological malignancy is a T-cell related tumor, which includes leukemia, lymphoma and myeloma.

The present invention also provides a method for treating/preventing cancer, comprising the step of administering a therapeutic effective amount of a drug to a subject in need, wherein the drug includes at least one of an antibody specifically binding to CD7 or the antigen binding fragment thereof, a separated nucleic acid, a carrier, a host cell, an anti-human CD7 chimeric antigen receptor CAR, anti-human CD7 chimeric antigen receptors CAR obtained from the preparation method for the anti-human CD7 chimeric antigen receptor CAR, an immunologic effector cell and immunologic effector cells obtained from the preparation method for the immunologic effector cell.

The terms “subject” and “patient” used in present invention are interchangeably used to refer to any animal that may require antibody related preparations or drugs or treatments described in present invention. The subjects and patients include but are not limited to, primates (including humans), canines, cats, mice and other mammalian subjects. Preferably, the subject is a human.

In the present invention, the term “treatment” refers to therapeutic treatment and preventive or prophylactic measures aimed at preventing or slowing down (reducing) unexpected physiological changes or disorders, such as the progression of autoimmune diseases. Beneficial or expected clinical outcomes include, but are not limited to the following either detectable or undetectable results: the relief of symptoms, the reduction in disease severity, the stability of disease status (i.e. not worsening), the delay or slowing of disease progression, the improvement or slowing of disease status and the alleviation (whether partial or complete). “Treatment” also refers to the extended survival period compared to the expected survival period when not receiving treatment. Those who need treatment include those who already have symptoms or disorders, as well as those who are prone to symptoms or disorders or those who need to prevent them.

The term “effective dose” used in present invention refers to the amount of drug or agent that triggers biological or pharmaceutical responses to such as tissues, systems, animals or humans pursued by researchers or clinical physicians, in. In addition, the term “therapeutic efficacy” refers to the amount of improved treatment, cure, prevention or reduction that causes disease, illness or side effects or a decrease in the rate of disease or condition progression, compared to the corresponding subjects who did not receive that amount. This term also includes the amount that effectively enhances normal physiological function within its scope. Usually, the effective dosage in present invention varies based on various factors, such as the given drug or compound, pharmaceutical formulation, route of administration, type of disease or illness, treated subjects, etc., but can still be routinely determined by those skilled in the art. The effective amount of the compound of the present invention can be easily determined by those skilled in the art through conventional methods known in the art.

The present invention also provides the application of the antibody or the antigen binding fragment thereof or the blocking molecule or the chimeric antigen receptor or the immunologic effector cell according to the present invention in combination with other drugs. Preferably, the other drugs include diagnostic agents, prophylactic agents and/or therapeutic agents. Furthermore, the other drugs are CD20 targeting antibody drugs, which include but are not limited to: rituximab, atozumab, oxfamumab, teimozumab, etc.

### Embodiment 1

This embodiment is the preparation of mouse monoclonal antibodies targeting the CD7 antigen.

This embodiment designs and synthesizes 5 peptides targeting CD7 antigen to immunize BALB/c mice. After cell fusion, initial screening and re-screening, one positive clone is obtained that simultaneously recognizes polypeptide BST001-2 and CD7 recombinant protein. The clone number of the positive hybridoma cell line is 5B5.

The amino acid sequence of the CD7 antigen precursor protein is shown in SEQ ID NO.: 1, wherein the amino acid residues 26-180 are the extracellular domain of the CD7 antigen. The CD7 recombinant protein is a recombinant human CD7 protein (labeled with his, product of Pepsius, item number 11028-H08H) and has an amino acid sequence of the CD7 antigen extracellular domain sequence as shown in SEQ ID NO.: 2.

The amino acid sequences of the 5 synthesized antigen polypeptides used for immunizing mice correspond to the 5 amino acid residues in the extracellular domain of the CD7 antigen, and the specific sequence is shown in Table 1.

TABLE 1
Amino acid sequences of 5 synthesized peptides

| Serial number | Sequences | Serial number in the |
|---|---|---|
| of polypeptides | of polypeptides | corresponding sequence table |
| BST001-1 | AQEVQQSPHC | SEQ ID NO: 3 |
| BST001-2 | CEDGVVPTTDRRFRGRID | SEQ ID NO: 4 |
| BST001-3 | TEEQSQGWHRC | SEQ ID NO: 5 |
| BST001-4 | HRLQLSDTGTYTC | SEQ ID NO: 6 |
| BST001-5 | CPPTGSALPD | SEQ ID NO: 7 |

Among the 5 polypeptides, the first amino acid at the N-terminus of BST001-2 and BST001-5 (bold in the table) is the added cysteine (Cys), while the amino acids at C-terminus of polypeptides BST001-1, BST001-3 and BST001-4 themselves are Cys. 5 polypeptides are conjugated with the carrier protein KLH (Keyhole Limpet Hemocyanin) through Cys residues at the N-terminus or C-terminus and used as immunogens to immunize a group of mice. The immunized mice are performed with the tail blood testing and the monoclonal antibody screening.

The mouse monoclonal antibody of the present invention is a monoclonal antibody that recognizes polypeptide BST001-2 and CD7 antigen proteins and obtained through immunization with the polypeptide BST001-2 as the immunogen. The following is a specific description using polypeptide BST001-2 as an example.

### 1.1 Immunity of Mice and the Immunoreaction Evaluation Thereof

BST001-2-KLH is mixed with adjuvants CFA and AD11.15 to prepare immunogens and immunize the three BALB/c mice. 14th days after immunization, the tail blood of the mice is extracted and the antibody titers in the serum are evaluated using indirect ELISA.

The specific operating steps of the indirect ELISA method are:
(1) coating the elisa plate by using BST001-2 polypeptides, each well is added with 100 μL of BST001-2 polypeptide (concentration 1 μg/mL) and reacted overnight at 4° C.;
(2) washing the plate three times with PBS solution and sealing it at room temperature for 1 hour with 5% Milk-PBS;
(3) washing the plate once with PBS solution, add mouse tail blood diluted with a gradient of 5% mill PBS solution and react at room temperature for 1 hour;
(4) washing the plate with PBS solution three times and pat dry and it is added with HRP labeled sheep-anti-mouse IgG (Fc) secondary antibody at a dilution of 1:2000 and reacted at room temperature for 1 hour;
(5) after washing the plate with PBS solution 5 times, patting it dry and adding an equal volume of A and B solutions and reacting it for 20 minutes under dark and room temperature conditions;
(6) adding 50 μL termination solution and mixed well and then reading the OD450 value on the microplate reader.

The indirect ELISA evaluation results of mouse tail blood 14th days after immunization are shown in Table 2. From the results, it can be seen that the tail blood antibody titers of all three mice have reached over 1:10000. Subsequently, 1 # mouse is selected for cell fusion in step 1.2 below.

TABLE 2
ELISA evaluation of antibody titers in mouse tail blood
14th days after immunization

| Mouse | Dilution ratio of mouse tail blood | | | | | |
|---|---|---|---|---|---|---|
| number | 1/500 | 1/1000 | 1/5000 | 1/10000 | 1/50000 | NC |
| 1# | 2.788 | 1.932 | 0.506 | 0.176 | 0.140 | 0.058 |
| 2# | 2.314 | 1.803 | 0.464 | 0.274 | 0.082 | 0.084 |
| 3# | 2.832 | 2.687 | 0.641 | 0.269 | 0.099 | 0.057 |

Note: The negative control NC is 5% Milk-PBS.

### 1.2 Cell Fusion and Screening of Hybridoma Cell Lines

According to the ELISA evaluation results of mouse tail blood in Table 2, on the 21st day after immunization, spleen cells of mice 1 # are selected to fuse with myeloma cells SP2/0. On the 10th day after fusion, 564 monoclonal cells are selected and cultured in a 96 well plate. After 7 days of cultivation, the supernatant of the 564 monoclonal cells cultured in the 96 well plate is evaluated using the indirect ELISA method described in step 1.1. Hybridoma cell lines capable of secreting monoclonal antibodies that recognize CD7 antigen peptides are screened. Seven positive clone strains are preliminarily selected from them.

Further re-screening experiments are conducted on the 7 positive clones using the indirect ELISA method described in step 1.1 and the results are shown in Table 3.

TABLE 3
ELISA re-screening confirmation of mouse fusion positive clones

| 7 positive clones | | | | | | | | |
|---|---|---|---|---|---|---|---|---|
| 2C5 | 3C2 | 3H5 | 5B5 | 6A5 | 6B5 | BG5 | NC | PC |
| 0.046 | 0.044 | 0.045 | 2.696 | 0.048 | 0.044 | 0.048 | 0.047 | 2.493 |
| 0.051 | 0.046 | 0.043 | 2.623 | 0.045 | 0.042 | 0.048 | 0.044 | 2.565 |

Note: The negative control NC is 5% Milk-PBS; The positive control PC is 1 # mouse cardiac blood, diluted at 1:500.

From the re-screening results in Table 3, a strongly positive hybridoma cell line 5B5 recognizing the BST001-2 polypeptide is obtained.

### 1.3 Reconfirmation of Strongly Positive Hybridoma Cell Line 5B5

Using the indirect ELISA method described in step 1.1, confirmation experiments are conducted again on the supernatant of the highly positive hybridoma clone strain 5B5 after proliferation culture. The results are shown in Table 4.

TABLE 4
ELISA double confirmation of 5B5 positive clones

| 5B5 | 5B5 | NC | NC | PC | PC |
|---|---|---|---|---|---|
| 2.677 | 2.749 | 0.316 | 0.107 | 2.767 | 2.677 |

Note: NC is a negative control 5% Milk-PBS; PC is used as a positive control for 1 # mouse cardiac blood, diluted at 1:500.

Further, by using the indirect ELISA method described in step 1.1, the CD7 recombinant protein (with his tag) is used instead of the BST001-2 polypeptide coated elisa plate to detect the cell supernatant of the 5B5 clone. The results are shown in Table 5.

TABLE 5
Competitive ELISA detection of 5B5 positive clones

| 5B5 | NC | PC |
|---|---|---|
| 2.782 | 0.047 | 2.636 |
| 2.759 | 0.043 | 2.629 |

Note: The negative control NC is 5% Milk-PBS; the positive control PC is an anti-CD7 recombinant protein mouse monoclonal antibody with His tag.

From the results in Tables 4 and 5 above, it can be seen that the cell supernatant of the 5B5 clone exhibits strong recognition reactions with the BST001-2 polypeptide antigen of CD7 and CD7 recombinant protein antigen. 5B5 is the desired hybridoma cell line that can produce anti-CD7 monoclonal antibodies.

### 1.4 Cloning and Sequencing of the Variable Region of Monoclonal Antibody Against Hybridoma Cell Line 5B5

5B5 hybridoma cell lines are cultured to collect cells and extract RNA so as to obtain cDNA sequences encoding the anti-CD7 monoclonal antibodies using RT-PCR. Then, the variable regions of the heavy and light chains using PCR are cloned and the PCR products are bound to the T-vector. The sequences of the heavy chain variable region VH and the light chain variable region VL in the anti-CD7 monoclonal antibody are sequenced, following by further comparing and confirming the sequences using the Uniprot database.

The nucleotide sequence of the obtained VH is shown in SEQ ID NO.: 8 and its encoding amino acid sequence is shown in SEQ ID NO.: 9; the nucleotide sequence of the obtained VL is shown in SEQ ID NO.: 10 and its encoding amino acid sequence is shown in SEQ ID NO.: 11.

Further analysis is conducted on the amino acid sequences of the obtained VH and VL to identify their complementary determining regions (CDR) and results are shown in Table 6.

| | | Amino acid sequence | Serial number |
|---|---|---|---|
| VH | CDR1 | GYSFTDY | SEQ ID NO: 12 |
| | CDR2 | DPYYGS | SEQ ID NO: 13 |
| | CDR3 | DGNYGSDY | SEQ ID NO: 14 |
| VL | CDR1 | RSSQSIVHSNGNTYLE | SEQ ID NO: 15 |
| | CDR2 | KVSNRFS | SEQ ID NO: 16 |
| | CDR3 | FQGSHVPFT | SEQ ID NO: 17 |

### Embodiment 2

This embodiment is the construction of CD7-Blocker and CD7-CAR lentivirus expression vectors.

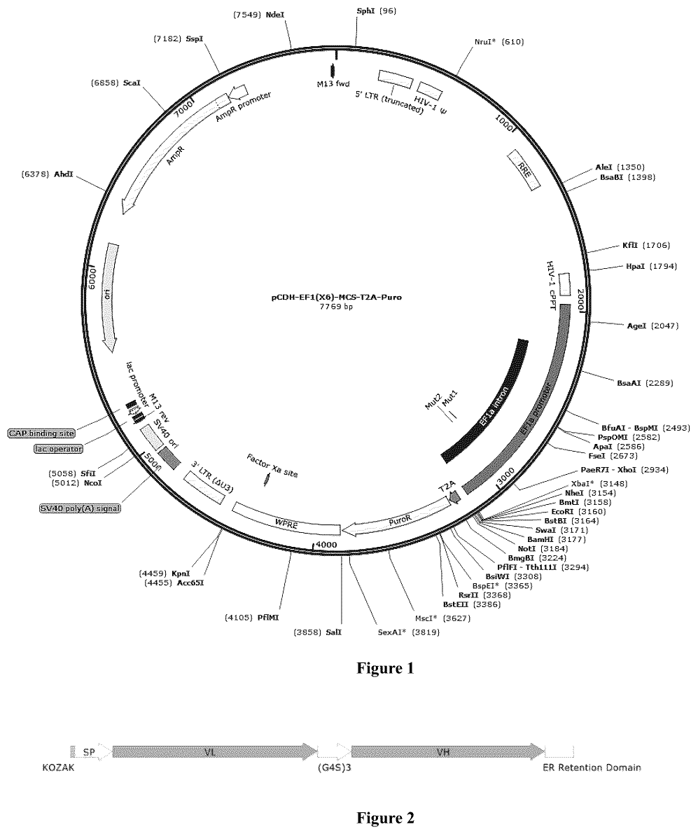

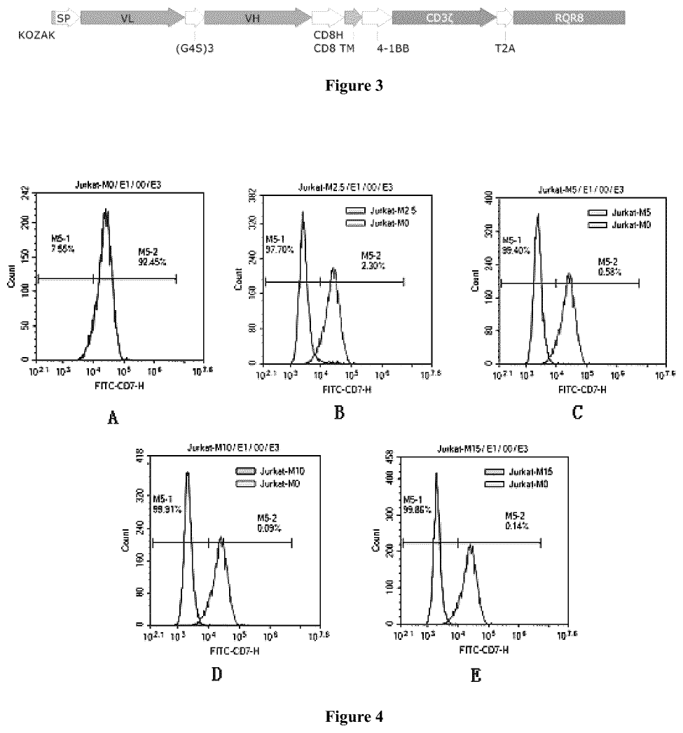

The CD7 blocking molecule CD7-Blocker and CD7-CAR are respectively constructed into third-generation lentivirus expression plasmids by using the conventional technical means in the art. The plasmid is pCDH-EF1 (X6)-MCS-T2A-Puro, and its map is shown in FIG. 1. The linearized cleavage sites of the vector are XbaI and SalI and the DNA sequences of CD7-Blocker and CD7-CAR (including N-terminal KOZAC sequences) are inserted between these two cleavage sites. The molecular structure of CD7-Blocker is shown in FIG. 2. The CD7-Blocker molecule consists of three parts: the CD8α signal peptide SP, the anti-CD7 scFv composed of VL and VH connected by a (G4S)3 linker and the ER Retention Domain (endoplasmic reticulum localization domain). The front end of SP is inserted with a KOZAK sequence that promotes expression. Its full-length amino acid sequence is shown in SEQ ID NO: 18, and its full-length DNA sequence is shown in SEQ ID NO: 19 (including N-terminal KOZAC sequence).

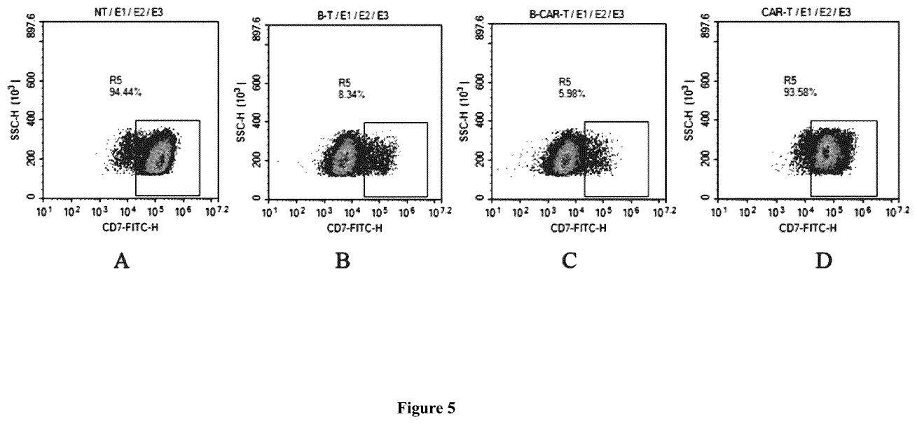

The molecular structure of CD7-CAR is shown in FIG. 3, which is composed of the CD8α signal peptide SP, the anti-CD7 scFv formed by (G4S)3 linker connecting VL and VH, the CD8α hinge region, the CD8α transmembrane region, the 4-1BB and CD3ζ two intracellular signaling domains form the main body of the CD7-CAR molecule, which is then fused with a cell “suicide switch” RQR8 molecule via an auto cleavage T2A linker peptide. The SP front end also inserts a KOZAK sequence that promotes expression. Its full-length amino acid sequence is shown in SEQ ID NO: 20, and its full-length DNA sequence is shown in SEQ ID NO: 21 (including N-terminal KOZAC sequence). The nucleotide coding sequences of VH and VL in the molecular structures of CD7-Blocker and CD7-CAR, as shown in SEQ ID NO: 22 and SEQ ID NO: 23, are optimized from mouse derived VH sequences (SEQ ID NO: 8) and VL sequences (SEQ ID NO: 10) according to human codons, respectively. VL and VH are fused through linker (G4S)3 to form anti-CD7 scFv.

The CD7-Blocker is formed by connecting the anti-CD7 scFv and ER localization signal KDEL through linker (G4S)2, and (G4S)2-KDEL forms the ER Retention Domain (endoplasmic reticulum localization domain). ER Retention Domain can localize anti-CD7 scFv to the ER reticulum. CD7 antigen molecules are intercepted by anti-CD7 scFv in the ER reticulum in the secretion pathway, so they cannot be properly localized to the cell surface, thereby avoiding the fratricide between CD7-CAR-T cells. In another embodiment, the linker connecting the anti-CD7 scFv to the ER localization signal is the myc sequence EQKLISEEDL (SEQ ID NO: 33).

The sequence numbers corresponding to the amino acid and nucleotide sequences of each fragment in the molecular structure of CD7-Blocker and CD7-CAR are shown in Table 7, wherein SP is the CD8α signal peptide, CD8H is the CD8α hinge region, CD8TM is the CD8α transmembrane region, 4-1BB and CD3 ζ All are intracellular signal transduction domains.

TABLE 7
Corresponding table of fragment sequences in CD7-Blocker and CD7-
CAR molecular structures

| Fragment Name | Amino Acid Sequence | Nucleotide Sequence |
|---|---|---|
| VH domain for anti-CD7 seFv | SEQ ID NO: 9 | SEQ ID NO: 22 |
| VL domain for anti-CD7 seFv | SEQ ID NO: 11 | SEQ ID NO: 23 |
| KOZAK | / | SEQ ID NO: 24 |
| (G4S)3 linker | SEQ ID NO: 25 | SEQ ID NO: 26 |
| ER positioning signal KDEL | SEQ ID NO: 27 | SEQ ID NO: 28 |
| (G4S)2 linker | SEQ ID NO: 29 | SEQ ID NO: 30 |
| ER Retention Domain | SEQ ID NO: 31 | SEQ ID NO: 32 |
| Myc linker | SEQ ID NO: 33 | SEQ ID NO: 34 |
| SP (CD8a Signal Peptide) | SEQ ID NO: 35 | SEQ ID NO: 36 |
| CD8H (CD8a hinge area) | SEQ ID NO: 37 | SEQ ID NO: 38 |
| CD8TM (CD8a transmembrane region) | SEQ ID NO: 39 | SEQ ID NO: 40 |
| 4-1BB | SEQ ID NO: 41 | SEQ ID NO: 42 |
| CD3ζ | SEQ ID NO: 43 | SEQ ID NO: 44 |
| T2A | SEQ ID NO: 45 | SEQ ID NO: 46 |
| RQR8 | SEQ ID NO: 47 | SEQ ID NO: 48 |
| CD20Epitope | SEQ ID NO: 49 | SEQ ID NO: 50 |

CAR-T cell therapy often accompanies various toxic side effects. In order to increase the safety of CAR-T cell therapy, the present invention incorporates a “suicide switch” RQR8 molecule (SEQ ID NO.: 47, SEQ ID NO.: 48) into the CD7-CAR molecular structure. The RQR8 molecule is fused to the intracellular signaling domain CD3ζ in the CD7-CAR structure by a T2A linker peptide with auto cleavage function. The RQR8 molecule carries two CD20 antigenic epitopes and can induce T cell apoptosis by targeting CD20 using anti-CD20 rituximab and activating the antibody dependent cell-transduced cytotoxicity (ADCC) and the complement transduced cytotoxicity (CDC). When necessary, the use of rituximab can achieve the elimination of CAR-T cells, thereby increasing the safety of CAR-T cell therapy.

The lentivirus packaging adopts the conventional four plasmid systems in the art, among which three auxiliary plasmids are pMDLg/pRRE, pRSV-Rev and pMD2.G. the 293T cell are used as lentiviral packaging cells. The plasmid dosage ratio of the lentivirus expression plasmids carrying CD7-Blocker or CD7-CAR, the 293T cell co-transfected with pMDLg/pRRE, pRSV-Rev and pMD2.G is 7.5:9:9:3.5; for T75 cell culture bottles, the four plasmid dosages are 7.5 μg, 9 μg, 9 μg and 3.5 μg, respectively. The dosage of transfection reagent PEI (μg) is three times of the total amount of four plasmids. For T75 culture bottles, the dosage of PEI is 87 μg (1 μg/μl, 87 μl).

48 hours after co-transfection of 293T with four plasmids, cell culture medium is collected, which is centrifuged (2000 rpm, 15 min) to obtain the supernatant. After the supernatant is filtered through a 0.45 μm filter, it is concentrated using ultracentrifugation (20000 rpm, 2 h). Then the virus precipitate is resuspended through the culture medium with the corresponding volume according to the dilution ratio, divided and stored at −80° C. for freezing.

For the CD7-Blocker lentivirus, the titer of the lentivirus is directly measured using the lentivirus vector HIV P24 rapid detection card. For the titer determination of CD7-CAR lentivirus, the lentivirus is performed with a series of gradient dilutions and transfected into the 293T cell. 48 hours later, the transfection efficiency is measured by flow cytometry to calculate the activity titer of the lentivirus.

### Embodiment 3

This embodiment is the affinity identification between Anti-CD7 scFv and CD7 antigen molecules.

CD7-CAR lentivirus is transduced with the 293T cell and flow cytometry is used to detect the positive rate of CD7-CAR in the 293T cell and the ratio of the 293T cell bound to CD7 antigen protein to calculate the affinity rate between CD7-CAR and CD7 antigen protein in CD7-CAR-the 293T cell thereby representing the affinity of Anti-CD7 scFv with CD7 antigen molecules.

The CD7 antigen protein is a recombinant human CD7 protein with His tag in Embodiment 1. During flow cytometry detection, CD7 antigen protein is first incubated with CD7-CAR-the 293T cell and then fluorescent labeled anti His mouse monoclonal antibody is used to detect CD7 antigen protein binding to the 293T cell.

Two flow cytometry analyses are performed on the 293T cell transduced by CD7-CAR lentivirus and the results are shown in Table 8.

TABLE 8
Affinity detection between Anti-CD7
scFv and CD7 antigen molecules

| | First detection | Second detection |
|---|---|---|
| Positive rate of | 71.38 | 70.21 |
| CD7-CAR in cells (%) | | |
| Positive rate of cells binding | 70.87 | 70.03 |
| with CD7 antigen molecules (%) | | |
| Affinity rate of the CD7 CAR and | 99.28 | 99.74 |
| the CD7 antigen molecule (%) | | |

| | |
|---|---|
| Average affinity | 99.51 |

The results in Table 8 show that the average affinity between CD7-CAR and CD7 antigen molecules is 99.51%, indicating that anti-CD7 scFv has a strong affinity with CD7 antigen molecules.

### Embodiment 4

This embodiment demonstrates the blocking effect of CD7-Blocker against CD7 molecules on the Jurkat cell surface.

The CD7-Blocker lentivirus is transfected into Jurkat cells. after 4 days later, the positive rate of CD7 molecules on the Jurkat cell surface is detected by flow cytometry. The results are shown in Table 9 and FIG. 4. The positive rate of CD7 molecules on the Jurkat cell surface without introducing CD7-Blocker is 92.45%; when the viral MOI=10, the positive rate of CD7 molecules on the Jurkat cell surface decreases to 0.09% (see FIG. 4D) and the blocking efficiency of D7 Blocker on the Jurkat cell surface reaches the limit value of 99.90%. When MOI=5 (see FIG. 4C), the blocking efficiency is approaching its limit and further increasing the MOI value to 15 (see FIG. 4E) has no significant effect on improving the blocking efficiency. These results indicate that CD7-Blocker can almost completely block the expression of CD7 molecules on the Jurkat cell surface.

TABLE 9
Inhibition efficiency of CD7-Blocker against
CD7 molecules on the Jurkat cell surface

| Transduction MOI | 0 | 2.5 | 5 | 10 | 15 |
|---|---|---|---|---|---|
| CD7 positivity rate (%) | 92.45 | 2.3 | 0.58 | 0.09 | 0.14 |
| CD7 positivity rate flow | FIG. 4A | FIG. 4B | FIG. 4C | FIG. 4D | FIG. 4E |
| cytometry | | | | | |
| CD7 blocking | | 97.5 | 99.37 | 99.90 | 99.89 |
| efficiency (%) | | | | | |

### Embodiment 5

This embodiment demonstrates the blocking effect of CD7-Blocker against CD7 molecules on the surface of T cells.

The frozen PBMC is resuscitated and activated by adding CD3/CD28 antibody magnetic beads (activation time point marked as D0). 1 day (D1) after activation, the CD7-Blocker lentivirus is transfected (B-T experimental group). 2 days (D2) after activation, the CD7-CAR lentivirus is transfected. The experiment is divided into four groups: non transfected control group (NT group), only transfected with CD7-Blocker virus (B-T group), co-transfected with CD7-Blocker and CD7-CAR virus on the basis of NT (B-CAR-T group), only transfected with CD7-CAR virus (CAR-T group). The MOI values for virus transfection are all 5. After transfection, the positive rate of CD7 antigen molecules on the cell surface is detected by flow cytometry at D6 (6 days after activation, 5 days after Blocker virus transfection and 4 days after CAR virus transfection) of transfection. The results are detailed in Table 10 and FIG. 5.

It can be seen that under the experimental conditions of this embodiment, the blocking efficiency of CD7-Blocker (B-T group) on the CD7 molecule reaches 91.12%, CD7-CAR alone (CAR-T group) has almost no effect on the cell surface expression of CD7 molecule and the blocking efficiency of CD7-Blocker&CD7-CAR (B-CAR-T group) on the CD7 molecule reached 93.67%.

TABLE 10
The blocking efficiency of CD7-Blocker
on the CD7 molecule T cell surface

| | NT | B-T | B-CAR-T | CAR-T |
|---|---|---|---|---|
| CD7 positivity rate (%) | 94.44 | 8.34 | 5.98 | 93.58 |
| CD7 positivity rate | FIG. 5A | FIG. 5B | FIG. 5C | FIG. 5C |
| flow cytometry | | | | |
| CD7 blocking efficiency (%) | | 91.12 | 93.67 | 0.91 |

### Embodiment 6

This embodiment illustrates the effect of CD7-Blocker on T cell proliferation.

This embodiment detected and compared the in vitro proliferation of four groups of T cells: control T cells (NT group) that are not transfected with CD7-Blocker virus, CD7-Blocker-T cells that are only transfected with CD7-Blocker lentivirus (B-T group), CD7-CAR-T cells that are only transfected with CD7-CAR lentivirus (CAR-T group) and The CD7-Blocker-CAR-T cell that are simultaneously transfected with CD7-Blocker virus and CD7-CAR virus (B-CAR-T group). The time points for virus transfection in each group of cells are as shown in Embodiment 4.

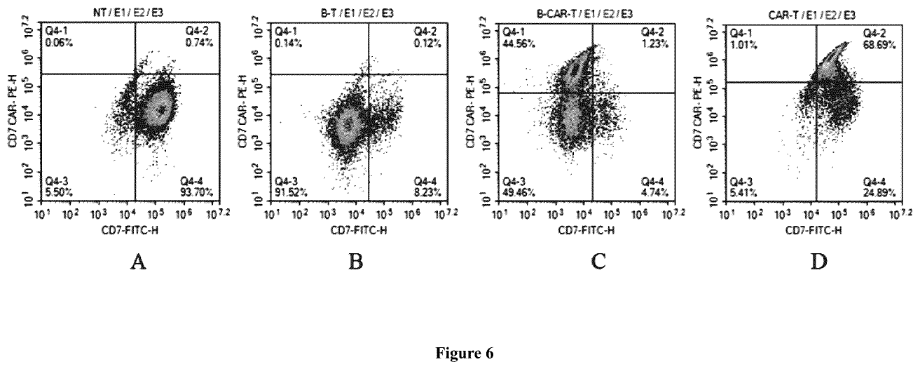

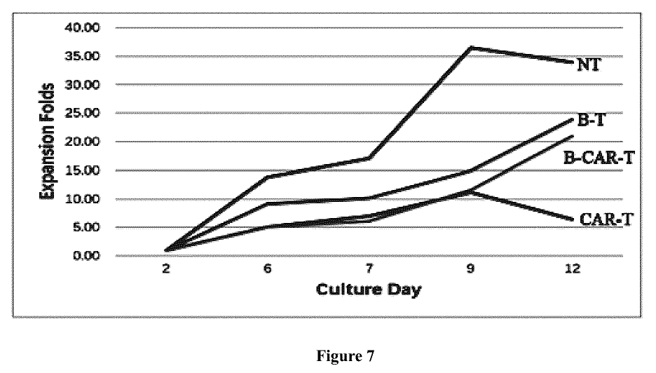

Cultivation time description: one day after thawing and cultivation of frozen PBMC, CD3/CD28 antibody magnetic beads are added for activation. The activation time point is recorded as D0 and the time points on the first and second day after activation are recorded as D1 and D2, respectively and so on. The surface CD7 molecule positivity rate and CAR positivity rate of four groups of T cells are shown in Table 11 and FIG. 6, the cell amplification ratio is shown in Table 12 and the cell amplification curve is shown in FIG. 7.

These results indicate that compared with control T cells, B-T cells transfected with CD7-Blocker virus alone and B-CAR-T cells co-transfected with CD7-Blocker virus and CD7-CAR virus can proliferate normally, while CAR-T cells transfected with only CD7-CAR lentivirus can hardly proliferate in vitro. This phenomenon indicates that blocking the CD7 molecule basically does not affect the normal proliferation of T cells, while CAR-T cells that are not blocked by the CD7 molecule can cause fratricide of CD7-CAR-T cells due to the CD7 expression on the surface.

TABLE 11
CD7 Molecular positive rate and CAR positive rate

| | NT | B-T | B-CAR-T | CAR-T |
|---|---|---|---|---|
| % CD7+ | 0.80 | 0.26 | 45.79 | 69.70 |
| % CAR+ | 94.44 | 8.35 | 5.97 | 93.58 |
| CD7 and CAR flow cytometry | FIG. 6A | FIG. 6B | FIG. 6C | FIG. 6C |
| detection map | | | | |

TABLE 12
Cell amplification multiples (relative to D 2)

| Day | 2 | 6 | 7 | 9 | 12 |
|---|---|---|---|---|---|
| NT | 1.00 | 13.85 | 17.04 | 36.46 | 33.91 |
| B-T | 1.00 | 9.12 | 10.12 | 14.88 | 23.81 |
| B-CAR-T | 1.00 | 5.08 | 6.05 | 11.62 | 20.92 |

### Embodiment 7

This embodiment is an in vitro killing experiment of CD7-CAR-T cells against CD7 positive target cells.

In order to further verify the specificity of CD7-CAR-T cells in killing CD7 positive target cells, a CD7 overexpressing U87-CD7-eGFP cell line is constructed using CD7 negative U87 cells. The killing effect of CD7-CAR-T cells on the CD7 positive target cell U87-CD7-eGFP is analyzed using RTCA instrument.

The CD7 antigen molecule encoding sequence used to construct the U87-CD7-eGFP cell line is the DNA encoding sequences of the CD7 antigen precursor protein (SEQ ID NO: 51). The amino acid sequence of the used eGFP molecule is shown in SEQ ID NO: 52, and its DNA encoding sequence is shown in SEQ ID NO: 53. The CD7 molecule is connected to the eGFP subunit by an auto cleavage linker peptide T2A (SEQ ID NO: 45, SEQ ID NO: 46). After adding KOZAK sequence to the N-terminus of CD7-T2A-eGFP structure, it is inserted between the XbaI and SalI two cleavage sites of the lentiviral vector pCDH-EF1 (X6)-MCS-T2A-Puro to construct a CD7 overexpression lentiviral vector. According to conventional methods, the lentivirus CD7-T2A-eGFP is transfected into U87 cells, and eGFP is used as a screening and detection marker for the transfected cells.

Due to the fratricide between CD7-CAR-T cells, the effector cells used in the killing experiment are CD7-Blocker-CAR-T cells (referred to as B-CAR-T) that are blocked by CD7 Blocker on the surface of CD7 molecules, with a CAR positive rate of 45.79% and a surface CD7 molecule positive rate of 5.97%. The control T cells for the killing experiment are CD7-Blocker-T (B-T) cells, with a surface CD7 molecule positivity rate of 8.35%. The positive rate of CD7 surface molecules on T cells without blockade is 94.44%. The above positive rate data is shown in Table 11 and FIG. 6 of Embodiment 6.

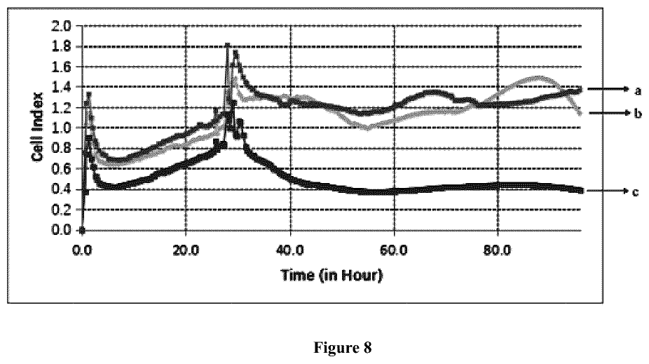

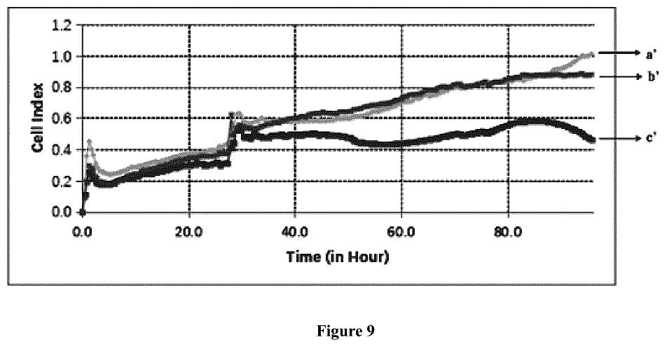

The killing experiment curves are shown in FIGS. 8 and 9. Time point 0.0 is the starting point of the target cell culture, and effector cells are added for co-culture 28 hours after the target cell culture. The co-culture effect target ratio is B-CAR-T (or control B-T): U87-CD7-eGFP (or control U87)=4:1. The entire experiment lasts for 96 hours.

FIG. 8 shows the killing curves of two types of T cells co-cultured with the positive target cell U87-CD7-eGFP. It can be seen that only B-CAR-T has a significant killing effect on CD7 positive target cell U87-CD7-eGFP. FIG. 9 shows the killing curves of two types of T cells co-cultured with the negative target cell U87. It can be seen that B-CAR-T has only a slight killing effect on U87. These results indicate that CD7-CAR-T cells blocked by CD7 Blocker have a significant and specific killing effect on CD7 positive target cells. The present invention provides beneficial CAR-T cells for the next clinical application of cell therapy.

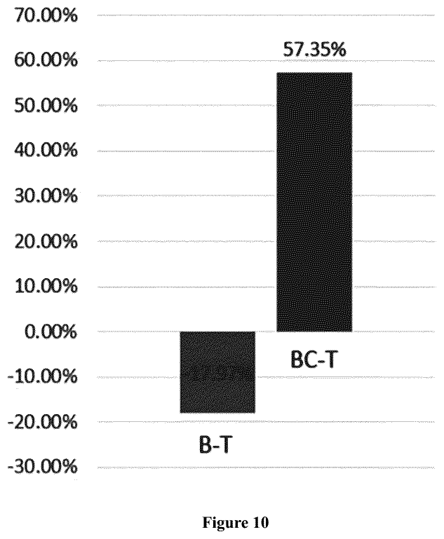

In order to further analyze the killing efficiency of B-CAR-T on target cells, the cell index values (Cell Index at: 60:04:20&at: 30:33:52) at both ends of the early time period of co-culture are extracted. The killing efficiency obtained is shown in FIG. 10. The B-T group is co-cultured with control CD7-Blocker-T cells and U87-CD7-eGFP cells, while the BC-T group is co-cultured with CD7 Blocker CAR-T cells and U87-CD7-eGFP cells. It can be seen that at E:T=4:1, compared with control B-T cells, B-CAR-T has a high killing efficiency against CD7 positive target cells.

Although the present invention has been described with reference to exemplary embodiments, it should be understood that the present invention is not limited to the disclosed exemplary embodiments. Multiple adjustments or variations may be made to the exemplary embodiments of the present invention without departing from the scope or spirit of the present invention. The scope of claims should be based on the widest interpretation to cover all modifications and equivalent structures and functions.

1. An antibody or the antigen binding fragment thereof comprising:
a heavy chain variable regions of antigen complementary determining regions CDR1, CDR2 and CDR3 with an amino acid sequences as shown in SEQ ID NO.: 12-14 respectively; and
a light chain variable region of antigen complementary determining regions CDR1, CDR2 and CDR3 with the amino acid sequence as shown in SEQ ID NO.: 15-17 respectively.

2. The antibody or the antigen binding fragment thereof according to claim 1, wherein the antibody has any one of the amino acid sequences as shown in (I), (II) or (III):
(I) a heavy chain variable region amino acid sequence as shown in SEQ ID NO.: 9 and a light chain variable region amino acid sequence as shown in SEQ ID NO.: 11;
(II) an amino acid sequences with at least 90%, preferably at least 95%, more preferably at least 98% and most preferably at least 99% homology to the amino acid sequences as shown in SEQ ID NO.: 9 and SEQ ID NO.: 11;
(III) an amino acid sequence obtained by subjecting the amino acid sequences as shown in SEQ ID NO.: 9 and SEQ ID NO.: 1 to modification, substitution, deletion or addition of one or more amino acids;
wherein, the amino acid sequence has an antibody activity against the tumor surface antigen CD7.

3. The antibody or the antigen binding fragment thereof according to claim 2, wherein the antibody comprises at least one of a polyclonal antibody, a monoclonal antibody, a chimeric antibody, a humanized antibody or a bispecific antibody; the antigen binding fragment includes at least one of a Fab fragment, a Fab′, a F(ab′)₂ fragment, a single chain variable fragment scFv, a scFv-Fc fragment or a single chain antibody ScAb.

4. A CD7 blocking molecule comprising:
a. the antibody or the antigen binding fragment thereof according to claim 1; and
b. an endoplasmic reticulum localization domain.

5. A chimeric antigen receptor comprising:
1) an antigen binding domain recognizing the CD7 antigen, wherein the antigen binding domain comprises an antibody or the antigen binding fragment thereof according to claim 1;
2) a transmembrane structural domain; and
3) an intracellular signal transduction domain;
preferably, the chimeric antigen receptor further comprises a hinge area;
preferably, the chimeric antigen receptor further comprises a suicide switch molecule;
preferably, the chimeric antigen receptor further comprises an intracellular costimulatory domain;
preferably, the transmembrane domain is selected from at least one peptides of CD28, NKp30, CDS, DAP10, 4-1BB, DAP12, CD3C, CD3ε, CD45, CD4, CD5, CD8, CD9, CD16, CD22, CD33, CD37, CD64, CD80, CD86, CD134, CD137, CD154, KIRDS2, OX40, CD2, CD27, LFA-1, ICOS (CD278), 4-1BB (CD137), GITR, CD40, BAFFR, HVEM (LIGHT), SLAMF7, NKp80 (KLRF1), CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAMI (CD226), SLAMF4 (CD244, 2B4), CD84, CD96, CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp or any combination thereof;
preferably, the intracellular signal transduction domain is selected from at least one of CD8, CD3ζ, CD3δ, CD3γ, CD3ε, FcγRI-γ, FcγRIII-γ, FceRIβ, FcεRIγ, DAP10, DAP12, CD32, CD79a, CD79b, CD28, CD3C, CD4, b2c, CD137 (4-1BB), ICOS, CD27, CD288, CD80, NKp30, OX40 or any combination thereof.

6. A separated nucleic acid molecule encoding the antibody or the antigen binding fragment thereof according to claim 1.

7. A carrier comprising the nucleic acid molecule according to claim 6.

8. A host cell comprising the carrier according to claim 7.

9. An immunologic effector cell expressing the antibody or the antigen binding fragment thereof according to claim 1, wherein
the immunologic effector cells are selected from at least one of a white blood cell, a monocyte, a macrophage, a dendritic cell, a mast cell, a neutrophil, a basophil, an eosinophil, an αβ T cell, a γδ T cell, a natural killer (NK) cell, a natural killer T (NKT) cell, a B cell, a natural lymphoid like cell (ILC), a cytokine induced killer (CIK) cell, a cytotoxic T lymphocyte (CTL), a lymphokine activated killer (LAK) cell, a T lymphocyte, a peripheral blood mononuclear cell and a hematopoietic stem cell.

10. An application of a reagent in the preparation of drugs for the prevention and/or treatment of cancer or tumors, wherein the reagent comprises the antibody or the antigen binding fragment thereof according to claim 1;
preferably, the cancer or tumor refers to a cancer or tumor associated with CD7 expression;
preferably, the cancer or tumor is a hematological malignancy;
further preferably, the hematological malignancy is a T-cell related tumor including leukemia, lymphoma and myeloma;
preferably, the application further includes the application of the antibody or the antigen binding fragment thereof in combination with other drugs;
preferably, the other drugs include a diagnostic agent, a prophylactic agent and/or a therapeutic agent;
preferably, the other drugs are drugs targeting the CD20 antibody.
